(19) United States
(12) Patent Application Publication
(10) Pub. No.: US 2016/0271059 A1
(43) Pub. Date: Sep. 22, 2016

(54) OPHTHALMIC EMULSION COMPOSITION OF CYCLOSPORINE

(71) Applicant: Gavis Pharmaceuticals, Somerset, NJ (US)

(72) Inventors: Aman Trehan, Somerset, NJ (US); Bala Chandran Nayar, Somerset, NJ (US)

(73) Assignee: GAVIS PHARMACEUTICALS, Somerset, NJ (US)

(21) Appl. No.: 14/659,345

(22) Filed: Mar. 16, 2015

(51) Int. Cl. A61K 9/107; A61K 47/44; A61K 38/13

(52) U.S. Cl. CPC A61K 9/107; A61K 38/13; A61K 47/44

(57) ABSTRACT

The invention relates to an ophthalmic emulsion composition of cyclosporine in the form of an oil-in-water emulsion. The emulsion remains stable at 25° C. for at least 6 months or at 45° C. for at least 45 days. The composition is useful for treating a dry eye condition or glaucoma, and preferably for increasing tear production in patients whose tear production is presumed to be suppressed due to ocular inflammation associated with keratoconjunctivitis sicca.

### BACKGROUND OF THE INVENTION

(a) Field of the Invention

Disclosed herein are ophthalmic emulsion compositions of cyclosporine in the form of oil-in-water emulsion. More specifically, the ophthalmic emulsion composition of cyclosporine comprises one or more anionic surfactants. The composition is useful for treating a dry eye condition or glaucoma, and preferably to increase tear production in patients whose tear production is presumed to be suppressed due to ocular inflammation associated with keratoconjunctivitis sicca.

(b) Description of the Related Art

Cyclosporines are a group of nonpolar cyclic oligopeptides with known immunosuppressant activity. In addition, as set forth in U.S. Pat. No. 4,839,342, cyclosporine (sometimes referred to in the literature as “cyclosporine”) has been found as effective in treating immune medicated keratoconjunctivitis sicca (KCS or dry eye disease) in a patient suffering therefrom.

As hereinabove noted, cyclosporine comprises a group of cyclic oligopeptides and the major component thereof is cyclosporine A (C₆₂H₁₁₁N₁₁O₁₂) which has been identified along with several other minor metabolites, cyclosporine B through I. In addition, a number of synthetic analogs have been prepared.

The chemical name of cyclosporine is cyclo[[(E)-(2S,3R,4R)-3-hydroxy-4-methyl-2-(methylamino)-6-octenoyl]-L-2-aminobutyryl-N-methylglycyl-N-methyl-L-leucyl-L-valyl-N-methyl-L-leucyl-L-alanyl-D-alanyl-N-methyl-L-leucyl-N-methyl-L-leucyl-N-methyl-L-valyl]. Cyclosporine has the following structure:

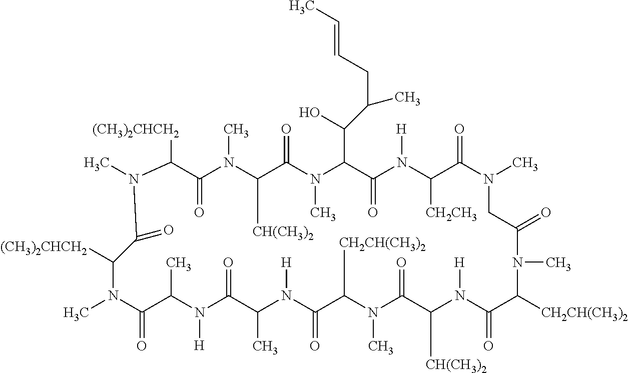

Dry eye disease is characterized by chronic drying of the conjunctiva and cornea, as well as by decreased tear production and changes in the composition of the tear film. In order to enhance the efficiency of cyclosporine A treatment, it becomes necessary to increase the absorption of the drug in the lachrymal gland as well as the conjunctiva and cornea target tissues, using for the purpose a suitable dosage of the drug to suppress ocular inflammation without significant systemic cyclosporine A exposure.

Since the aqueous solubility of cyclosporine A is between about 20 to 30 μg/ml, there is no adequate aqueous formulations available for ocular administration of the drug. Moreover, if cyclosporine is administered orally for the treatment of KCS, the accompanying side effects due to systemic circulation may cause adverse reactions such as hypertrichosis or renal dysfunction. Due to the limited solubility and hydrophobic nature of the drug, the use of cyclosporine A in oral formulations is limited.

U.S. Pat. No. 4,839,342 discloses cyclosporine as a group of nonpolar cyclic oligopeptides with known immunosuppressant activity and has been found as effective in treating immune medicated keratoconjunctivitis sicca (KCS or dry eye disease) in a patient suffering therefrom.

The first ophthalmic emulsion of cyclosporine was commercially available in the United States at a 0.05% concentration under the brand name Restasis®. The product appears to be white opaque to a slightly translucent homogeneous emulsion with an osmolality of 230 to 320 mOsmol/kg, a pH of 6.5-8.0 and containing Polysorbate 80 as a surfactant. The emulsion has an overall anionic nature.

Restasis® ophthalmic emulsion is indicated to increase tear production in patients whose tear production is presumed to be suppressed due to ocular inflammation associated with keratoconjunctivitis sicca.

Problems associated with cyclosporine and its derivatives are due to their rather poor water solubility. Consequently many different ophthalmic formulations have been proposed to overcome this problem.

WO 2001005819 discloses a water-soluble cyclosporine conjugated compound in which cyclosporine is chemically bound to a water-soluble polymeric or macromolecular carrier that renders the drug water-soluble and more bioavailable.

U.S. Pat. No. 5,051,402 discloses that since the solubility of cyclosporine in water was extremely low, it has been considered not merely difficult but practically impossible to prepare a pharmaceutical composition containing cyclosporine dissolved in an aqueous medium.

U.S. Pat. No. 5,474,979 discloses a pharmaceutical composition in the form of a non-irritating emulsion which includes at least one cyclosporine in admixture with a higher fatty acid glyceride and polysorbate 80, a non-ionic surfactant. More particularly, the cyclosporine may be cyclosporine A and the higher fatty acid glyceride may be castor oil.

U.S. Pat. No. 6,582,718 discloses an ophthalmic composition particularly in the form of eye-drops suitable for the treatment of diseases of the eye and surrounding areas. The composition contains a cyclosporine and a non-ionic surfactant selected from polyoxyethylene fatty acid esters, polyoxyethylene alkylphenyl ethers and polyoxyethylene alkyl ethers, or mixtures thereof.

U.S. Application No. 20130323270 discloses a Cremophor-free cyclosporine composition. Particularly, the publication discloses a cyclosporine emulsion containing a long chain triglyceride (a natural oil), a phosphatidylcholine, glycerol, a pharmaceutically tolerable alkali salt of a free fatty acid, a medium chain triglyceride-oil and water.

U.S. Pat. Nos. 8,629,111; 8,633,162; 8,642,556; 8,648,048; and 8,685,930 disclose use of cyclosporine emulsion compositions including water, a hydrophobic component and a cyclosporine component in a therapeutically effective amount of less than 0.1% by weight of the composition. The weight ratio of the cyclosporine component to the hydrophobic component is less than 0.8. The patents teach the use of non-ionic surfactants, such as polysorbate 80, and that the overall nature of the emulsion is anionic.

U.S. Pat. No. 6,656,460 discloses cyclosporine emulsion compositions for treating a dry eye condition by topically applying to the eye surface an emulsion for forming a tear film that adheres electrostatically to the entire surface of the eye and acts to lubricate the eye and inhibit evaporation of moisture therefrom. The formulation disclosed in the patent contains poloxamer as the emulsifying agent, and the overall nature of the emulsion is cationic.

U.S. Pat. No. 8,298,568 discloses oil-in-water emulsions useful as a delivery vehicle of hydrophobic ingredients such as cyclosporine. According to the '568 patent, the emulsion particles have a net positive charge and comprise 0.001 to 0.1% of a cationic agent, 0 to 1% of a non-ionic surfactant and 0 to 0.5% of an anionic surfactant. The overall nature of the emulsion is also cationic.

In spite of the various ophthalmic emulsions disclosed in the prior art there still exists a need to develop an alternate, stable ophthalmic emulsion composition of cyclosporine that is safe for use in treating a dry eye condition or glaucoma, and preferably to increase tear production in patients with ocular inflammation associated with keratoconjunctivitis sicca.

### SUMMARY OF THE INVENTION

The present invention provides an ophthalmic emulsion composition of cyclosporine. Particularly, the ophthalmic oil-in-water emulsion composition of cyclosporine comprises an anionic surfactant. Essentially, the emulsion is devoid of any non-ionic surfactant.

In one aspect, the invention provides an ophthalmic oil-in-water emulsion composition consisting essentially of:
(a) cyclosporine A in an amount of about 0.001% to about 0.1% by weight;
(b) castor oil or sesame oil in an amount of about 0.01% to about 1.5% by weight;
(c) at least one anionic surfactant; and
(d) water,
   wherein, the emulsion is devoid of a non-ionic surfactant.

In another aspect, the invention provides an ophthalmic oil-in-water emulsion composition consisting essentially of:
(a) cyclosporine A in an amount of about 0.001% to about 0.1% by weight;
(b) castor oil or sesame oil in an amount of about 0.01% to about 1.5% by weight;
(c) at least one anionic surfactant in an amount of about 0.001% to about 0.5% by weight;
(d) at least one viscosity enhancing agent; and
(e) water,
   wherein, the emulsion is devoid of a non-ionic surfactant.

In another aspect, the invention provides an ophthalmic oil-in-water emulsion composition comprising:
(a) cyclosporine A in an amount of about 0.05% by weight;
(b) castor oil or sesame oil in an amount of about 1.25% by weight;
(c) at least one anionic surfactant in an amount of about 0.001% to about 0.5% by weight;
(d) at least one viscosity enhancing agent; and
(e) water,
   wherein, the emulsion is devoid of non-ionic surfactant.

In another aspect, the ophthalmic oil-in-water emulsion is devoid of any additional oil, preferably devoid of a medium chain triglyceride oil, such as coconut oil or palm oil.

In another aspect, the ophthalmic oil-in-water emulsion composition of the invention further comprises one or more cationic surfactants.

In another aspect, the ophthalmic oil-in-water emulsion remains stable at 25° C. for at least 6 months or at 45° C. for at least 45 days.

In another aspect, the ophthalmic oil-in-water emulsion is characterised by having neutral, positive or negative zeta potential. Preferably the emulsion has a neutral zeta potential.

In another aspect, the ophthalmic oil-in-water emulsion composition of the invention is characterised by having a positive zeta potential in the range of between 70 mV and 30 mV.

In another aspect, the ophthalmic oil-in-water emulsion composition of the invention is characterised by having a negative zeta potential in the range of between −1 mV and −50 mV.

In another aspect, ophthalmic oil-in-water emulsion composition of the invention is characterised by having a substantially neutral zeta potential, preferably a zeta potential between −10 mV and +10 mV, more preferably between −4 mV and +4 mV, and still more preferably between −2 mV and +2 mV.

In another aspect, the weight ratio of the cyclosporine to castor oil or sesame oil in the ophthalmic oil-in-water emulsion composition of the invention is less than 0.8.

The ophthalmic oil-in-water emulsion composition of the invention comprises one or more pharmaceutically acceptable ingredients selected from the group comprising oils, lipids, polyelectrolytes, viscosity modifying agents, demulcents, pH modifying agents, and tonicity adjusting agents, etc.

In another aspect, the pH of the ophthalmic oil-in-water emulsion composition of the invention is in the range of about 5.0 to about 9.0, preferably about 7.2 to about 7.6.

In another aspect, the colloidal particles of the ophthalmic oil-in-water emulsion composition of the invention have an average particle size of about 100 to about 500 nm.

The ophthalmic oil-in-water emulsion composition of the invention is suitable for single use, or repeated or periodic, multiple use in the form of eye drops or a similar form or other form so as to facilitate such administration to the eye.

In another aspect, the invention provides a method of increasing tear production in patients whose tear production is presumed to be suppressed due to ocular inflammation associated with keratoconjunctivitis sicca. The method comprises application of an ophthalmic oil-in-water emulsion as substantially described herein and above to an eye of said patient.

Still other aspects and advantages of the invention will be apparent from the following detailed description of the invention.

### DETAILED DESCRIPTION OF THE INVENTION

The subject matter of the present invention is an ophthalmic oil-in-water emulsion composition comprising cyclosporine and one or more anionic surfactants. The overall emulsion is devoid of any non-ionic surfactant. Essentially the composition is devoid of any additional oil, preferably devoid of medium chain triglyceride oil, such as coconut oil or palm oil.

It has now been unexpectedly found that a combination of an oily phase containing a cyclosporine, an aqueous phase, and a surfactant component which is an anionic surfactant can generate a stable emulsion for ophthalmic use. These emulsions may have positive, negative or a substantially neutral electrochemical charge (zeta potential) and have excellent stability characteristics both in terms of physical properties of the emulsion and the chemical stability of the cyclosporine.

One of the approaches to stabilize an emulsion is by conferring an electrostatic charge to the droplet surface which will result in droplet repulsion and less droplet coalescence. Depending on the nature of the film substances, the external surface of the colloid particles may be charged. Colloidal particles dispersed in a solution are electrically changed due to their ionic characteristics and dipole attributes. This charge, which can be negative resulting in anionic emulsions or positive producing cationic emulsions (Klang et al., Pharm. Dev. Technology 2000, 5, 521-532) is known in the art as the “zeta potential” and is a measure of the magnitude of the repulsion or attraction between particles.

It was found that by using only an anionic surfactant and avoiding the use of a non-ionic surfactant and medium chain triglyceride oil, a stable emulsion that remains stable at 25° C. for at least 6 months or at 45° C. for at least 45 days can be obtained. The stability of such is comparable to that of emulsions available commercially or otherwise known in the prior art.

The term “Cyclosporine A” as used in the invention is meant to cover crystalline Cyclosporine or Cyclosporine A in the form of freebase or its pharmaceutically acceptable salts, hydrates, solvates and physiologically functional derivatives and precursors thereof.

Embodiments are directed to an ophthalmic emulsion composition of cyclosporine that comprises cyclosporine and one or more anionic surfactants. The emulsion is devoid of any non-ionic surfactant.

Preferably, the embodiments are directed to an ophthalmic oil-in-water emulsion composition consisting essentially of:
(a) cyclosporine A in an amount of about 0.001% to about 0.1% by weight;
(b) castor oil or sesame oil in an amount of about 0.01% to about 1.5% by weight;
(c) at least one anionic surfactant; and
(d) water,
   wherein, the emulsion is devoid of a non-ionic surfactant.

The anionic surfactants according to present invention include phospholipids. Examples of phospholipids, which may be used in the emulsions of the invention, are lecithins; Epikuron® (mixture of about 70% phosphatidylcholine and 12% phosphatidylethanolamine and about 15% other phospholipids); Ovothin 106® or Ovothin 200® (phosphatidylcholine, 18% phosphatidylethanolamine and 12% other phospholipids); a purified phospholipid mixture for example obtained from egg yolk; Lipoid E-80® (phospholipid mixture comprising about 80% phosphatidylcholine, 8% phosphatidylethanolamine, 3.6% non-polar lipids and about 2% sphingomyeline), alkyl sulfates include ammonium lauryl sulfate, sodium lauryl sulfate/sodium dodecyl sulfate and related alkyl-ether sulfates, sodium laureth sulfate, sodium myreth sulfate, sodium laurate, sodium stearate, potassium stearate, sodium oleate, and docusates. Docusates includes dioctyl sodium sulfosuccinate, perfluorooctane sulfonate (PFOS), perfluorobutanesulfonate, linear alkylbenzene sulfonates, Sodium Cocoyl Isethionate, Sodium Cocoyl Glutamate, and combinations thereof. Preferably, the anionic surfactant is selected from Sodium Cocoyl Isethionate and/or Sodium Cocoyl Glutamate.

The concentration of the anionic surfactant is preferably in an amount of between 0.001 to 0.05%.

The concentration of cyclosporine A and castor oil or sesame oil is preferably in an amount of about 0.05% and about 1.25% by weight, respectively.

In an embodiment, the ophthalmic oil-in-water emulsion compositions have a weight ratio of the cyclosporine to the castor oil or sesame oil of less than 0.8. The large amount of castor oil or sesame oil relative to the amount of cyclosporine in emulsion results in rapidly breaking down or resolving the emulsion in the eye and reduces vision distortion, as well as facilitating the therapeutic effectiveness of the composition in treating dry eye disease.

Preferably, the emulsion is characterized by having a positive zeta potential, negative zeta potential, or substantially neutral zeta potential. Preferably the emulsion is characterized by having a substantially neutral zeta potential. In one embodiment, the emulsion has a positive zeta potential in the range of between 70 mV and 30 mV. In a further embodiment, the emulsion has a negative zeta potential in the range of between −1 mV and −50 mV. In a further embodiment, the emulsion is characterised by having a substantially neutral zeta potential, preferably a zeta potential between −10 mV and +10 mV, more preferably between −4 mV and +4 mV, and still more preferably between −2 mV and +2 mV.

Zeta potential measurements may be carried out by various methods known in the art. In a method carried out with the Zetasizer NanoZS of the Malvern Instruments Ltd (UK), the sample (0.75 ml) to be analyzed was placed, by using a syringe to avoid bubbles, in a folded capillary cell fitted with electrodes. The cell is inserted in the instrument and the measurement sequence is started automatically; all the individual measurement runs are accumulated together and then summed to give the final Zeta potential result. It is not necessary to dilute the samples for the analysis and consequently there was no risk of artificially changing the characteristics of the sample.

The ophthalmic oil-in-water emulsion composition according to the present invention has pH in the range of about 5.0 to about 9.0, preferably 7.2 to about 7.6.

In a further embodiment, the emulsion droplet (or globule or particle) of the ophthalmic oil-in-water emulsion composition has an average particle size of about 100 nm to about 500 nm.

In another embodiment, the ophthalmic emulsion composition can be a single use or multiple, periodic use in the form of eye drops or a similar form or other form so as to facilitate such topical administration.

In an embodiment, the ophthalmic oil-in-water emulsion composition of the invention further comprises one or more cationic surfactants.

Cationic surfactants according to the present invention include but are not limited to C₁₀-C₂₄ primary alkylamines, tertiary aliphatic amines, quaternary ammonium compounds, cationic lipids, amino alcohols, chlorhexidine salts, cationic polymers and mixtures thereof.

The primary amine is selected from the group oleyl amine and stearyl amine; the tertiary aliphatic salt can be dimethyl lauramine or diethanolamine, the amino alcohol can be tris (hydroxymethyl) aminomethane, the chlorhexidine salt can be chlorhexidine dihydrochloride, the cationic polymer can be chitosan, the cationic lipid can be 1,2-dioleyl-3-trimethylammonium-propane, 1,2-dioleoyl-sn-glycero-phosphatidylethanolamine, cationic glycosphingo-lipids or cationic cholesterol derivatives and the quaternary ammonium compound can be selected from the group consisting of benzalkonium chloride, cetrimide, hexadecyltrimethylammonium bromide or chloride, tetracyltrimethylammonium bromide or chloride, dodecyltrimethylammonium bromide or chloride, cetrimonium chloride, benzethonium chloride or bromide, behenalkonium chloride or bromide, bis-2-hydroxyethyl oleyl amine, cetalkonium chloride or bromide, cetethyldimonium chloride or bromide, cetylpyridinium chloride or bromide, dipalmitoyl hydroxyethylmonium methosulfate, distearyldimonium chloride or bromide, stearalkonium chloride or bromide, myrtrimonium chloride or bromide, benzododecinium chloride or bromide, chlorallyl methenamine, 1-propanaminium, 3-(D-gluconoylamino)-N-(2-hydroxyethyl)-N,N-dimethylchloride and minkamidopropyl dimethyl 2-hydroxyethyl ammonium chloride or bromide and mixtures thereof.

The concentration of the cationic surfactant is an amount of between 0.001 to 0.05% and concentration of the anionic surfactant, if present, is preferably in an amount of between 0.001 to 0.05%.

The emulsion of the present invention may include one or more other pharmaceutically acceptable excipients in amounts effective to facilitate the usefulness and effectiveness of the compositions. Examples of such include without limitation, hydrophobic components, tonicity agent, polyelectrolyte, viscosity modifying agent, demulcent, emulsifier, acids and/or bases to adjust the pH of the composition, buffer, preservative and the like. Agents may be employed which are effective to perform two or more functions in the presently useful compositions. For example, one agent which is effective as both emulsifiers and surfactants can be employed, and/or components which are effective as both polyelectrolyte and viscosity modifying components can be employed.

The hydrophobic components, in addition to castor oil or sesame oil, may include, without limitation, olive oil, arachis oil, mineral oil, silicone fluid and the like and mixtures thereof. Preferably the composition is devoid of medium chain triglyceride oils, such as coconut oil or palm oil. Higher fatty acid glycerides such as castor oil, sesame oil, olive oil, peanut oil, and the like and mixtures thereof are particularly useful in the present invention. The hydrophobic components may be present in an amount in the range of about 0.001% to about 1.5% by weight of the emulsion composition.

Suitable viscosity modifying agents may include, but are not limited to, cellulose polymers, including hydroxypropyl methyl cellulose (HPMC), hydroxyethyl cellulose (HEC), ethyl hydroxyethyl cellulose, hydroxypropyl cellulose, methyl cellulose and carboxymethyl cellulose; carbomers (e.g. carbopol, and the like); polyvinyl alcohol; polyvinyl pyrrolidone; alginates; carrageenans; and guar, karaya, agarose, locust bean, tragacanth and xanthan gums. Such viscosity modifying components are employed, if at all, in an amount effective to provide a desired viscosity to the present compositions. The concentration of such viscosity modifiers will typically vary between about 0.01 to about 5% w/v of the total composition, although other concentrations of certain viscosity modifying components may be employed.

Suitable polyelectrolyte or emulsion stabilizing agents include, but are not limited to anionic cellulose derivatives, anionic acrylic acid-containing polymers, anionic methacrylic acid-containing polymers, anionic amino acid-containing polymers and the like and mixtures thereof. The polyelectrolyte may be present in an amount in a range of about 0.01% to about 1% by weight, preferably in the range of about 0.02% to about 0.5% by weight, of the composition.

Suitable demulcents can be included in effective amounts in the presently useful compositions. For example, suitable ophthalmic demulcent components include carboxymethylcellulose, other cellulose polymers, dextran 70, gelatin, glycerine, polyethylene glycols, propylene glycol, polyvinyl alcohol, povidone and the like and mixtures thereof. The demulcent may be in a range of about 0.01% to about 1.0% by weight, preferably from about 0.02% to about 0.5% by weight of the composition.

Suitable preservative according to the invention may include, but are not limited to, benzalkonium chloride, methyl and ethyl parabens, hexetidine, phenyl mercuric salts and the like and mixtures thereof. The amounts of preservative components included in the present compositions are such as to be effective in preserving the compositions and can vary based on the specific preservative component employed, the specific composition involved, the specific application involved, and like factors. Preservative concentrations often are in the range of about 0.00001% to about 0.05% or about 0.1% by weight of the composition, although other concentrations of certain preservatives may be employed.

It is generally understood by the person skilled in the art that substances having surfactant and preservative properties (e.g. benzalkonium chloride) are used in particular amounts for their intended purposes as either a preservative or a surfactant.

The oil-in-water emulsion composition of the invention may be suitable for single use, or periodic multiple use in the form of eye drops or similar form or other form so as to facilitate such application to an eye.

The emulsions may be prepared by a process comprising the steps: (a) optionally solubilising the formulation agents in the aqueous phase, (b) solubilising the anionic surfactant either in the aqueous or oily phase, (c) solubilising the cyclosporine in the oily phase, and (d) mixing the oily phase with the aqueous phase.

Embodiments are also directed to a method of increasing tear production in patients whose tear production is presumed to be suppressed due to ocular inflammation associated with keratoconjunctivitis sicca. The method comprises administration of an ophthalmic oil-in-water emulsion as substantially described herein throughout the specification to an eye of said patient.

### Example 1

### Cyclosporine Ophthalmic Emulsion

TABLE 1

| Sr. | | Formula 1 | Formula 2 | Formula 3 |
|---|---|---|---|---|
| No. | Ingredients | (% w/v) | (% w/v) | (% w/v) |
| 1 | Cyclosporine | 0.05 | 0.05 | 0.05 |
| 2 | Sesame Oil or Soybean Oil | 1.25 | 2 | 2.5 |
| 3 | Sodium Cocoyl Isethionate or | 0.1 | 0.2 | 0.3 |
| | Sodium Cocoyl Glutamate | | | |
| 4 | Sodium Hydroxide or | Q.S. | Q.S. | Q.S. |
| | Hydrochloric Acid | | | |
| 5 | Sodium Chloride | Q.S. | Q.S. | Q.S. |
| 6 | Purified Water | Q.S. | Q.S. | Q.S. |

Procedure:

Sodium Chloride and Sodium Cocoyl Isethionate/Sodium Cocoyl Glutamate were dissolved in water to prepare an aqueous solution. Separately, a dispersion of cyclosporine in Sesame Oil/Soybean Oil was made and added to the aqueous solution to form an oil-in-water emulsion by high shear mixing. The desired pH of the emulsion was adjusted with sodium hydroxide or hydrochloric acid.

1. An ophthalmic oil-in-water emulsion composition consisting essentially of:
(a) cyclosporine A in an amount of about 0.001% to about 0.1% by weight;
(b) an oil comprising castor oil, sesame oil or a combination in an amount of about 0.01% to about 1.5% by weight;
(c) at least one anionic surfactant; and
(d) water, wherein, the emulsion is devoid of a non-ionic surfactant.

2. The ophthalmic oil-in-water emulsion of claim 1, wherein said emulsion is characterised by being devoid of medium chain triglyceride oils.

3. The ophthalmic oil-in-water emulsion of claim 1, wherein the cyclosporine A is present in an amount of about 0.05% by weight.

4. The ophthalmic oil-in-water emulsion of claim 1, wherein said oil is present in an amount of about 1.25% by weight.

5. The ophthalmic oil-in-water emulsion of claim 1, said anionic surfactant comprises phospholipids, ammonium lauryl sulfate, sodium lauryl sulfate, sodium laurate, sodium stearate, potassium stearate, sodium oleate, sodium laureth sulfate, sodium myreth sulfate, dioctyl sodium sulfosuccinate, perfluorooctanesulfonate, perfluorobutanesulfonate, linear alkylbenzene sulfonates, Sodium Cocoyl Isethionate, Sodium Cocoyl Glutamate, or combinations thereof.

6. The ophthalmic oil-in-water emulsion of claim 1, wherein said anionic surfactant is Sodium Cocoyl Isethionate, Sodium Cocoyl Glutamate, or combinations thereof.

7. The ophthalmic oil-in-water emulsion of claim 1, wherein said emulsion further contains one or more cationic surfactants.

8. The ophthalmic oil-in-water emulsion of claim 1, wherein said emulsion is characterised by having a negative zeta potential.

9. The ophthalmic oil-in-water emulsion of claim 8, wherein said positive zeta potential is in the range of between 70 mV and 30 mV.

10. The ophthalmic oil-in-water emulsion of claim 8, wherein said negative zeta potential is in the range of between −1 mV and −50 mV.

11. The ophthalmic oil-in-water emulsion of claim 1, wherein said emulsion is characterised by having a substantially neutral zeta potential.

12. The ophthalmic oil-in-water emulsion of claim 11, wherein said neutral zeta potential is in the range of between −10 mV and 10 mV.

13. The ophthalmic oil-in-water emulsion of claim 1, wherein the droplet of said emulsion has an average particle size of about 100 nm to about 500 nm.

14. The ophthalmic oil-in-water emulsion of claim 1, wherein the pH of said emulsion is in the range of about 5.0 to about 9.0.

15. The ophthalmic oil-in-water emulsion of claim 1, wherein said emulsion remains stable at 25° C. for at least 6 months or at 45° C. for at least 45 days.

16. The ophthalmic oil-in-water emulsion of claim 1, wherein the composition consists of:
(a) cyclosporine A in an amount of about 0.001% to about 0.1% by weight;
(b) an oil comprising castor oil, sesame oil or a combination in an amount of about 0.01% to about 1.5% by weight;
(c) at least one anionic surfactant; and
(d) water, wherein, the emulsion is devoid of a non-ionic surfactant.

17. A method of increasing tear production in patients whose tear production is presumed to be suppressed due to ocular inflammation associated with keratoconjunctivitis sicca, said method comprises application of the ophthalmic oil-in-water emulsion of claim 1 to the eye of said patient.

18. The ophthalmic oil-in-water emulsion of claim 1, wherein the composition consists of:
(a) cyclosporine A in an amount of about 0.05% weight/volume;
(b) sesame oil in an amount of about 1.25% weight/volume;
(c) sodium cocoyl isethionate or sodium cocoyl glutamate as the at least one anionic surfactant in an amount of about 0.1-0.3% weight/volume;
(d) sodium hydroxide or hydrochloric acid to adjust the pH;
(e) sodium chloride; and
(f) water,
wherein, the emulsion is devoid of a non-ionic surfactant.

19. The ophthalmic oil-in-water emulsion of claim 18, wherein said emulsion is characterized by having a negative zeta potential in the range of between −1 mV and −50 mV.

20. An ophthalmic oil-in-water emulsion composition, wherein the composition consists of:
(a) cyclosporine A in an amount of about 0.05% weight/volume;
(b) sesame oil or soybean oil in an amount of about 1.25% to about 2.5% weight/volume;
(c) sodium cocoyl isethionate or sodium cocoyl glutamate in an amount of about 0.1% to about 0.3% weight/volume;
(d) sodium hydroxide or hydrochloric acid in an amount necessary to adjust the pH to a range of about 5.0 to about 9.0;
(e) sodium chloride; and
(f) water,
wherein, the emulsion is devoid of a non-ionic surfactant, and the emulsion has a negative zeta potential in the range of between −1 mV and −50 mV.
